(19) United States
(12) Patent Application Publication
(10) Pub. No.: US 2026/0140025 A1
(43) Pub. Date: May 21, 2026

(54) Aquatic Radionuclide Monitoring System

(71) Applicant: ATOMIC ENERGY OF CANADA LIMITED/ÉNERGIE ATOMIQUE DU CANADA LIMITÉE, Chalk River (CA)

(72) Inventors: Volodymyr KOROLEVYCH, Chalk River (CA); Dan FESTARINI, Chalk River (CA); Kim VU, Chalk River (CA); David GRAND-MAITRE, Chalk River (CA); Luke LEBEL, Chalk River (CA)

(21) Appl. No.: 19/119,659

(22) PCT Filed: Oct. 13, 2023
(86) PCT No.: PCT/CA2023/051362

§ 371 Date: Apr. 9, 2025

Related U.S. Application Data
(60) Provisional application No. 63/415,722, filed on Oct. 13, 2022.

(51) Int. Cl. G01N 1/40; G01N 33/18; G01T 1/36

(52) U.S. Cl. CPC G01N 1/4005; G01N 33/1893; G01T 1/366; G01N 2001/4016

(57) ABSTRACT

A flow-though system and method are provided for measuring radionuclides in an aqueous solution. The method includes: continuously directing a source stream of an aqueous solution into a combined stream; continuously directing a concentrate stream exiting a concentration loop into the combined stream, and wherein the concentration loop comprises a detection portion; continuously directing the combined stream into a reverse osmosis unit and separating the combined stream using the reverse osmosis unit to output the concentrate stream and a filtrate stream; continuously directing the concentrate stream exiting the reverse osmosis unit into the concentration loop to be recirculated into the combined stream upon exiting the concentration loop; and obtaining a measurement of the radionuclides at the detection portion using a spectrometer.

### CROSS-REFERENCE TO RELATED APPLICATION(S)

This patent application claims priority to U.S. Provisional Patent Application No. 63/415,722, filed on Oct. 13, 2022 and entitled “AQUATIC RADIONUCLIDE MONITORING SYSTEM”, the entire contents of which are herein incorporated by reference.

### FIELD OF THE INVENTION

In at least one of its aspects, the present disclosure relates generally to a system and method for monitoring aquatic radionuclides.

### INTRODUCTION

Japanese patent publication no. JP2008209396A describes a continuous concentrating apparatus for an analyzer capable of being applied to an analyzer which has reached its detection limit, and continuously obtaining objects to be analyzed having a fixed concentration ratio. The concentrating apparatus has a concentration loop which is a closed loop, wherein a sample liquid which is brought to flow in through a sample liquid supply port, pressurized by a pump and separated into a permeable liquid and a concentrated liquid by an RO unit, and the concentrated liquid is returned to the pump again by a check valve. The ratio of the permeable liquid flow rate to the concentrated liquid flow rate, obtained by taking a portion of the concentrated liquid out of the concentrating loop A, is controlled, thereby obtaining the concentrated liquid having the desired concentration ratio.

M. Vasyanovich et al./Nuclear Engineering and Technology 53 (2021) 253-257 describes the use of a baromembrane method based on reverse osmosis (RO) process. The method is realized on a mobile complex, which allows it to concentrate and determine ultra-low activity of radionuclides in water cooling ponds of Russian nuclear fuel cycle enterprises. The existence level of radionuclide background creates difficult conditions for identification of the contribution of liquid discharges enterprise, as standard monitoring methods have a very high detection level for radionuclides. Traditional methods for determining the background radionuclides concentrations require the selection of at least 500 liters of water, followed by their evaporation to form a dry residue. This procedure with RO membranes requires at least 5 days. It is possible to reduce the time and energy spent on evaporation of hundreds of water liters by pre-concentrating radionuclides in a smaller sample volume with baromembrane method. In some cases, this approach allows preliminary concentration of water samples from 500 liters volume till 20 liters volume during several hours. This approach is universal for the concentration of dissolved salts of any heavy metals, other organic compounds and allows the preparation of water countable samples in much shorter time compared to the traditional evaporation method.

A severe accident at a nuclear power plant can potentially emit dozens of different radionuclide species. The most important releases from the Fukushima accident were the radioisotopes of Xe, I, Te, Cs, Tc, La, Sb, Ba, Ag, for example. The direct emission of contaminated water that occurred during the Fukushima Daiichi accident has been well documented. The general contamination in the ocean was characterized in the year after the accident. It was postulated that observations of ⁹⁰Sr and ⁸⁹Sr in seawater off coast from the damaged plant occurred due to direct aquatic releases because strontium releases through the atmospheric route were not observed. Later, a better estimate of the fraction of cesium that was directly discharged through the aquatic route was made by correlating ¹³⁴Cs in seawater it to short lived radium isotopes (whose source is the coastline). This study estimated about 1.1×10¹⁶ Bq of both ¹³⁴Cs and ¹³⁷Cs through the direct aquatic discharge route, with the peak releases occurring in early April 2011, about a month after the accident. Other estimates, computed via inverse modeling, put the estimates of ¹³⁴Cs and ¹³⁷Cs releases via direct effluent discharge at about 3.5×10¹⁵ Bq, and at about 1.1×10¹⁶ Bq for ¹³¹I. Direct aquatic releases did not stop, though, as there was a prolonged period of groundwater ingress into the reactor building sumps and leakage back out into the environment, requiring several sets of controls to attempt to contain the contaminated groundwater, such as an underground ice wall to prevent the migration of groundwater to the sea, and replacing surrounded vegetation with asphalt surfaces to prevent rainwater ingression. Estimated discharges into the port of the Fukushima Daiichi Nuclear Power Plant started above 10¹⁵Bq/month just after the accident, down to between 10¹¹-10¹²Bq/month after the first year, and then plateaued between 10¹⁰-10¹¹Bq/month in the time span between 2014 and 2018. The ecological impacts and transport characteristics of the radiological discharges to the aquatic environment continue to be a part of active study.

In the event of an accident, it is important to have suitable monitoring and screening technologies in place for any impacted water bodies, preferably one that can be applied in the field. In some cases, the monitoring and screening technologies should specifically be able to measure radionuclides in the range of concentration of, for example, operation intervention levels (OIL) based on those recommended by the IAEA (International Atomic Energy Agency) concentrations values. In some cases, the monitoring and screening technologies are able to measure concentrations values in the range of 100 Bq/L to 1000 Bq/L, but also in the orders of magnitude above and below this.

A method developed to measure low-level radionuclides in Russian nuclear power plant cooling ponds used a mobile system that could be set up a cooling pond. Reverse osmosis (RO) membranes are used as a closed system with step-by-step volume decreases due to removal of permeate from the system (Vasyanovich et al. Nuclear Engineering and Technology, Volume 53, Issue 1, 2021, Pages 253-257). The method allowed a 30-40 times concentration of radionuclides in water using RO membranes, while the initial sample volume could be reduced from 1000 to 30 liters. The resulting sample was then sent to the lab and evaporated for sample analysis. While an improvement time-wise and could sample at site, it still required large sample sizes and laboratory work. It is not suitable for dynamic situations such as a nuclear accident.

JP2008209396A discloses a continuous sampling procedure that uses reverse osmosis with a recycle loop to continuously obtain an analyte concentrated by a constant concentration ratio. It does not specify how to apply such a process to measuring low-level radionuclides.

There remains a need for both a rapid and continuous method for evaluating low-level water-borne radionuclide concentrations.

### SUMMARY

This summary is intended to introduce the reader to the more detailed description that follows and not to limit or define any claimed or as yet unclaimed invention. One or more inventions may reside in any combination or sub-combination of the elements or process steps disclosed in any part of this document including its claims and figures.

In a first example broad aspect, a flow-though method for measuring radionuclides in an aqueous solution is provided. The method comprises:
continuously directing a source stream of an aqueous solution, comprising a first concentration of radionuclides, into a combined stream;
continuously directing a concentrate stream exiting a concentration loop into the combined stream, wherein the concentrate stream comprises a second concentration of the radionuclides that is greater than the first concentration of the radionuclides, and wherein the concentration loop comprises a detection portion;
continuously directing the combined stream into a reverse osmosis unit and separating the combined stream using the reverse osmosis unit to output the concentrate stream and a filtrate stream, wherein the filtrate stream comprises a third concentration of the radionuclides that is less than the second concentration of the radionuclides;
continuously directing the concentrate stream exiting the reverse osmosis unit into the concentration loop to be recirculated into the combined stream upon exiting the concentration loop; and
obtaining a measurement of the radionuclides at the detection portion using a spectrometer.

In some cases of the flow-through method, the spectrometer repeatedly measures the radionuclides at the detection portion in near real-time.

In some cases, the flow-through method further comprises: obtaining a salinity ratio representing a relationship between a first salinity of the concentrate stream and a second salinity of the source stream; and computing, using a controller, a value for the first concentration of radionuclides present in the source stream using at least the salinity ratio and the measurement of radionuclides at the detection portion.

In some cases of the flow-through method, a first sensor module is positioned in the concentration loop and repeatedly measures a first salinity and a first flow of the concentrate stream; and a second sensor module is positioned upstream from the reverse osmosis unit and repeatedly measures a second salinity and a second flow of the source stream prior to entering the reverse osmosis unit.

In some cases, the flow-through method further comprises:
a controller obtaining a value of the first salinity from the first sensor module and a value of the second salinity from the second sensor module;
the controller computing a salinity ratio using the value of the first salinity and the value of the second salinity; and
the controller computing a value for the first concentration of radionuclides present in the source stream using at least the salinity ratio and the measurement of radionuclides at the detection portion.

In some cases of the flow-through method, a third sensor module is positioned downstream from the reverse osmosis unit and repeatedly measures a third salinity and a third flow of the filtrate stream outputted by the reverse osmosis unit.

In some cases of the flow-through method, the third concentration of the radionuclides in the filtrate stream is less than the first concentration of the radionuclides in the source stream.

In some cases of the flow-through method, the third concentration of the radionuclides in the filtrate stream is about equal to the first concentration of the radionuclides in the source stream.

In some cases of the flow-through method, the obtaining the measurement of the radionuclides at the detection portion occurs while the concentrate stream is flowing through the detection portion.

In some cases of the flow-through method, the spectrometer is a gamma spectrometer.

In some cases of the flow-through method, the gamma spectrometer is a cadmium zinc telluride (CZT) semiconductor-based device.

In some cases of the flow-through method, the spectrometer is at least partially positioned within the detection portion.

In some cases, the flow-through method further comprises detecting that a steady state of total dissolved solids in the concentration loop is achieved.

In some cases, the flow-through method further comprises transmitting radioactivity data using a communication interface, wherein the radioactivity data comprises the measurement of the radionuclides, or the radioactivity data is derived from the measurement of the radionuclides.

In some cases, the flow-through method further comprises outputting radioactivity data via a user I/O device, wherein the radioactivity data comprises the measurement of the radionuclides, or the radioactivity data is derived from the measurement of the radionuclides.

In some cases of the flow-through method, the radioactivity data comprises an alert indicating that the measurement of the radionuclides is above a threshold value.

In some cases, the flow-through method further comprises:
performing the method of the first example at a first time and obtaining a first instance of the measurement of the radionuclides, corresponding to the first time;
computing a first instance value of the first concentration of radionuclides, corresponding to the first time, using at least the first instance of the measurement of the radionuclides;
repeating the method of the first example at a second time and obtaining a second instance of the measurement of the radionuclides, corresponding to the second time, wherein the second time occurs within a predetermined time period after the first time;
computing a second instance value of the first concentration of radionuclides, corresponding to the second time, using at least the second instance of the measurement of the radionuclides;
comparing the first instance value of the first concentration of radionuclides, to the second instance value of the first concentration of radionuclides; and
triggering an alert after detecting that the second instance value of the first concentration of radionuclides, has increased from the first instance value of the first concentration of radionuclides, by at least a given threshold value.

In some cases, the flow-through method further comprises:
performing the method of the first example at a first time and obtaining a first instance of the measurement of the radionuclides, corresponding to the first time;
computing a first instance value of the first concentration of radionuclides, corresponding to the first time, using at least the first instance of the measurement of the radionuclides;
triggering an alert after detecting that the first instance value of the first concentration of radionuclides is greater than a given threshold value.

In some cases, the flow-through method further comprises discarding the filtrate stream outputted by the reverse osmosis unit.

In some cases, the flow-through method further comprises controlling an amount of the concentrate stream being recirculated in the concentration loop using a pressure valve that is in fluidic communication with the concentration loop.

In a second example broad aspect, a flow-through system for monitoring radionuclides in aqueous solution is provided. The flow-through system comprises:
a flow-through system input configured to receive a source stream of an aqueous solution;
a reverse osmosis unit comprising a reverse osmosis input, a reverse osmosis output, and a concentrate output;
a detection portion comprising a detection portion input and a detecting portion output;
a spectrometer positioned at the detection portion;
a combining portion comprising a combining input and a combining output; and
a flow-through system output;
wherein:
   the combining input is in fluidic communication with the flow-through system input and the detection portion output, and is configured to receive the source stream from the flow-through system input and a concentrate stream outputted by the detecting portion output;
   the combining output is configured to output a combined stream that comprises the source stream and the concentrate stream outputted by the detecting portion output;
   the reverse osmosis input is in fluidic communication with the combining output and is configured to receive the combined stream;
   the reverse osmosis unit is configured to separate the combined stream into the concentrate stream and a filtrate stream, output the concentrate stream via the concentrate output, and output the filtrate stream via the reverse osmosis output;
   the concentrate output is in fluidic communication with the detection portion input and the detection portion input is configured to receive the concentrate stream outputted by the concentrate output;
   the detection portion output is configured to recirculate the concentrate stream into the combined stream;
   the spectrometer is operable to obtain a measurement of radionuclides in an amount of the concentrated stream within the detection portion; and
   the flow-through system output is in fluidic communication with the reverse osmosis output and is configured to output the filtrate stream.

In some cases, the flow-through system further comprises a pump operable to: continuously flow the source stream through the flow-through system input, continuously flow the combined stream into the reverse osmosis input, continuously flow the concentrate stream through the detection portion and recirculate the concentrate stream back into the combined portion, and continuously flow the filtrate stream out the flow-through system output.

In some cases of the flow-through system, the pump comprises a pump input and a pump output; the pump input is in fluidic communication with the combining output and receive the combined stream; and the pump output is in fluidic communication with the reverse osmosis input and outputs the combined stream to the reverse osmosis input.

In some cases of the flow-through system, the source stream comprises a first concentration of the radionuclides, and further wherein the reverse osmosis unit comprises a membrane configured to separate the combined stream into the concentrate stream that comprises a second concentration of the radionuclides that is greater than the first concentration of the radionuclides, and the filtrate stream that comprises a third concentration of the radionuclides that is less than the second concentration of the radionuclides.

In some cases, the flow-through system further comprises a first sensor module positioned to measure the concentrate stream, and the first sensor module operable to measure a first salinity and a first flow of the concentrated stream.

In some cases of the flow-through system, the first sensor module is positioned downstream from the detection portion output.

In some cases, the flow-through system further comprises a second sensor module positioned to measure the source stream, and the second sensor module operable to measure a second salinity and a second flow of the source stream.

In some cases, the flow-through system further comprises a third sensor module positioned to be in contact with the filtrate stream, and the third sensor module operable to measure a third salinity and a third flow of the filtrate water stream.

In some cases, the flow-through system further comprises a controller that is in data communication with at least the first sensor module and the spectrometer, and is configured to transmit radioactivity data associated with the concentrated stream.

In some cases of the flow-through system, the controller comprises memory that stores executable instructions, the executable instructions comprising:
obtain the measurement of radionuclides in the amount of the concentrated stream within the detection portion; and
compute a radioactivity value corresponding to the source stream by at least applying a scaling factor to the measurement of radionuclides corresponding to the concentrated stream.

In some cases of the flow-through system, the scaling factor comprises a salinity ratio between the concentrate stream and the source stream.

In some cases of the flow-through system, the executable instructions further comprise: detecting a steady state associated with at least total dissolved solids in the concentrated stream.

In some cases of the flow-through system, the spectrometer is a gamma spectrometer.

In some cases of the flow-through system, the gamma spectrometer is a cadmium zinc telluride (CZT) semiconductor-based device.

In some cases of the flow-through system, the detection portion comprises a vessel defining therein a vessel interior through which the amount of the concentrate stream in the detection portion flows through, and the spectrometer is at least partially positioned within the vessel interior.

In some cases, the flow-through system further comprises a pressure valve that is operable to interact with the concentrated stream, including controlling a volume of the concentrated stream that is recirculated back into the combining portion.

In some cases of the flow-through system, the pressure valve is positioned downstream from the detection output and upstream from the combining input.

In some cases, the flow-through system further comprises an overflow valve that is operable to interact with the concentrate stream, and an input of the overflow valve is positioned upstream from an input of the pressure valve.

In some cases, the flow-through system further comprises a body, and at least the reverse osmosis unit, the detection portion, the spectrometer, and the combining portion are supported by the body.

In some cases of the flow-through system, the body is portable and one or more wheels are mounted to the body.

In some cases, the flow-through system is portable.

### BRIEF DESCRIPTION OF THE DRAWINGS

Embodiments of the present invention will be described with reference to the accompanying drawings, wherein like reference numerals denote like parts, and in which:

FIG. 1 is a schematic representation of a flow-though system for measuring radionuclides in an aqueous solution operable to being operated in a continuous mode, according to at least some example embodiments;

FIG. 2 is a schematic diagram representation of a controller and a power supply coupled to one or more of the components in the aquatic radionuclide monitoring system, according to at least some example embodiments;

FIG. 3 is a schematic diagram representation of a controller's components, according to at least some example embodiments;

FIG. 4 is a schematic diagram representation of a reverse osmosis unit, according to at least some example embodiments;

FIG. 5 is a flow diagram for a flow-through method for measuring radionuclides in an aqueous solution, according to at least some example embodiments;

FIG. 6 is a plot showing the approximate gamma attenuation distance as a function of energy, according to at least some example embodiments;

FIGS. 7A and 7B are respectively a line drawing and a Computer Aided Design (CAD) rendering of one example of a detection portion that holds aqueous solution and a gamma spectrometer positioned at least partially within the interior of the detection portion, according to at least some example embodiments;

FIG. 8 is a CAD rendering of one example of a system including a reverse osmosis unit, a detection, a gamma ray spectrometer and a concentration loop, according to at least some example embodiments;

FIG. 9 is a photograph of a prototype of an aquatic radionuclide sampler during field testing, Nov. 25, 2021, according to an example embodiment;

FIG. 10A shows a geometry of a detection portion, according to at least some example embodiments, and FIG. 10B shows a cross-section view taken along the line Y-Y as illustrated in FIG. 10A. A volume of water is held within the body of the detection portion. A polycarbonate holder is positioned within the water, and an interior space is defined within the holder. A spectrometer is positioned within the interior space of the holder, surrounded by air that is within the interior space of the holder.

FIG. 11 is a plot showing the overall efficiency as a function of energy for various gamma lines from the isotope decays, according to an example embodiment. Not all lines are included, only ones with a statistical error of less than 30%;

FIG. 12 is a plot showing soluble activities in Node 9 (decay corrected to 14 days) for a single-unit accident in the multi-unit 4×878 MWe station for the SBO unmitigated and delayed onset cases, according to an example embodiment;

FIG. 13 is a plot showing soluble activities in Node 9 (decay corrected to 14 days) for a single-unit accident in a multi-unit 4×878 MWe station for the SBO with accident mitigation cases, according to an example embodiment; and

FIG. 14 is a flow diagram for another flow-through method for measuring radionuclides in an aqueous solution, according to at least some example embodiments.

### DETAILED DESCRIPTION

Various apparatuses or processes will be described below to provide an example of an embodiment of each claimed invention. No embodiment described below limits any claimed invention and any claimed invention may cover processes or apparatuses that differ from those described below. The claimed inventions are not limited to apparatuses or processes having all of the features of any one apparatus or process described below or to features common to multiple or all of the apparatuses described below. It is possible that an apparatus or process described below is not an embodiment of any claimed invention. Any invention disclosed in an apparatus or process described below that is not claimed in this document may be the subject matter of another protective instrument, for example, a continuing patent application, and the applicants, inventors, or owners do not intend to abandon, disclaim, or dedicate to the public any such invention by its disclosure in this document.

The Fukushima accident was compounded by a large earthquake and tsunami, and contaminated water in the reactor building sumps were technically outside of containment. It is difficult to know exactly if the same type of groundwater ingress and exchange with the environment would occur with contaminated water in a CANDU reactor containment, without further detailed analysis. Should some of this water leak out in the vicinity of drinking water sources, e.g. into the Great Lakes, it is important to have suitable monitoring and screening technologies in place for any impacted drinking water supply.

In some cases, analyses of the potential direct aquatic release source terms has been conducted in the context of confirming drinking water operational intervention levels (OILs). In some cases, it was confirmed that the existing OIL in use in Canada, based on those recommended by the IAEA, would be applicable for CANDU accidents and direct aquatic releases, as long as the list of IAEA OIL7 (food post-analysis) marker radionuclides were expanded to include 4 in total:
¹³¹I with a concentration OIL of 1000 Bq/L
¹³⁷Cs with a concentration OIL of 200 Bq/L
¹⁰⁶Ru with a concentration OIL of 100 Bq/L
⁸⁹Sr with a concentration OIL of 300 Bq/L

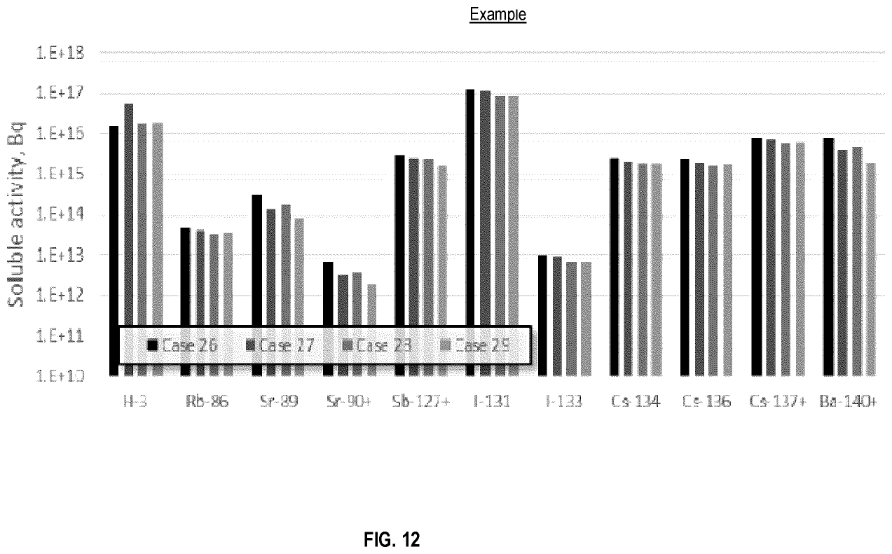

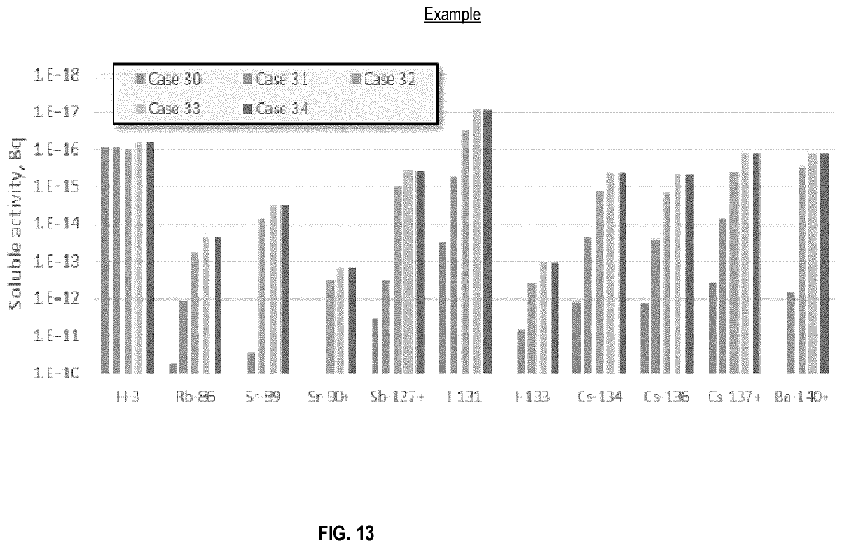

Moreover, the analysis of the potential direct aquatic release source terms concluded that the set of soluble radionuclides shown in FIG. 12 would be available in the water sumps.

A flow-through system has been developed for measuring radionuclides from a source stream of aqueous solution. In some cases, aqueous solution includes water, such as obtained from outflow of power plant, a stream, a lake, an ocean, a well, or another underground water source. It will be appreciated that one or more substances may be dissolved in the water.

In some cases, the flow-through system monitors gamma-emitting radionuclides in water in near real-time. In some cases, the flow-through system is configured to and operable to measure waterborne radioactivity concentrations in the environment during a accident in real-time.

In some cases, the flow-through system measures waterborne radioactivity concentrations in the environment during an accident. In some cases, the flow-through system measures radionuclides in the range of concentration of the OIL7 concentrations values above, so essentially in the range of 100 Bq/L to 1000 Bq/L, but also in the orders of magnitude above and below this.

Gamma spectrometers have a fairly wide dynamic measurement range before becoming saturated. It is recognized by the inventors named in this patent application that, in some cases, gamma spectrometers are unlikely to be sensitive enough to detect radionuclides in the water directly (or at least this would restrict the detection limits), but that the sensitivity could be improved by concentrating the dissolved radionuclides in the water. The flow-through system and the flow-through method provided herein include a reverse osmosis system, where aqueous solution and the radionuclides in the aqueous solution are recirculated.

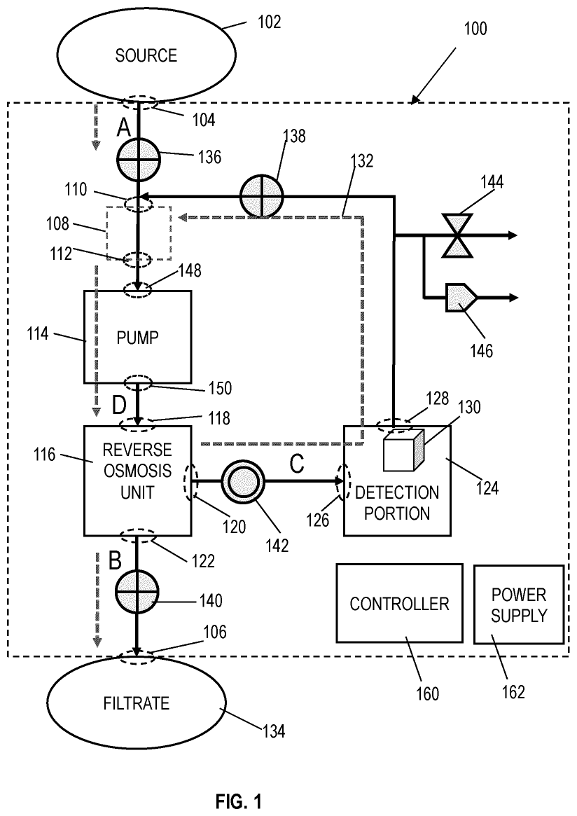

Referring to FIG. 1, an example of a flow-through system 100 is provided. The flow-through system is configured for monitoring radionuclides in aqueous solution coming from a source 102. Examples of the source 102 of aqueous solution include an outflow of a power plant, a stream, a lake, an ocean, a pond, a reservoir, a well, another underground water source, a tank, a pipe, etc. More generally, the source 102 of aqueous solution may be a naturally occurring source or a manmade source.

The flow-through system 100 includes a flow-through system input 104 configured to receive a source stream A of an aqueous solution. The source stream A, for example, comes from the source 102. The flow through system 100 also includes a flow-through system output 106. The flow-through system 100 further includes a combining portion 108 that includes a combining input 110 and a combining output 112. The flow-through system 100 further includes a reverse osmosis unit 116 that includes a reverse osmosis input 118, a reverse osmosis output 122, and a concentrate output 120. The flow-through system 100 further includes a detection portion 124 comprising a detection portion input 126 and a detecting portion output 128. A spectrometer 130 is positioned at the detection portion 124.

In some cases, the combining input 110 is in fluidic communication with the flow-through system input 104 and the detection portion output 128, and is configured to receive the source stream A from the flow-through system input 104 and a concentrate stream C outputted by the detecting portion output 128. The combining output 112 is configured to output a combined stream D that comprises the source stream A and the concentrate stream C outputted by the detecting portion output 128. The reverse osmosis input 118 is in fluidic communication with the combining output 112 and is configured to receive the combined stream D. The reverse osmosis unit 116 is configured to separate the combined stream D into the concentrate stream C and a filtrate stream B, output the concentrate stream C via the concentrate output 120, and output the filtrate stream B via the reverse osmosis output 122. The concentrate output 120 is in fluidic communication with the detection portion input 126 and the detection portion input 126 is configured to receive the concentrate stream C outputted by the concentrate output 120. The detection portion output 128 is configured to recirculate the concentrate stream C into the combined stream D. The spectrometer 130 is operable to obtain a measurement of radionuclides in an amount of the concentrated stream C within the detection portion 124. The flow-through system output 106 is in fluidic communication with the reverse osmosis output 122 and is configured to output the filtrate stream B.

In some cases, the source 102 is a large body of aqueous solution or is a moving stream, and the filtrate 134 that has been discarded from the flow-through system 100 is outputted or returned back to a different location within the source 102 compared to a location in the source 102 that the flow-through system input 104 receives or obtains the source stream A. In some cases, the flow-through system input 104 includes an intake tube that is placed at a first location within the source 102, and the flow-through system output 106 includes an outlet tube that is placed at a second location within the source, whereby the second location is different from the first location. In some cases, the intake tube, or more generally the flow-through system input 104, includes a filter to reduce or prevent solids from entering the flow-through system 100 as solids may clog one or more of the components in the flow-through system. In some other cases, the filtrate 134 is stored separately from the source 102.

In some cases, the flow-through system 100 comprises a pump 114 that is operable to continuously flow the source stream A through the flow-through system input 104, continuously flow the combined stream D into the reverse osmosis input 118, continuously flow the concentrate stream C through the detection portion 124 and recirculate the concentrate stream C back into the combined portion 108, and continuously flow the filtrate stream B out the flow-through system output 106.

In some cases of the flow-through system 100, the pump 114 comprises a pump input 148 and a pump output 150. In some cases, the pump input 148 is in fluidic communication with the combining output 112 and receives the combined stream D, and the pump output 150 is in fluidic communication with the reverse osmosis input 118 and outputs the combined stream D to the reverse osmosis input 118. In some other cases, the pump 114 is positioned elsewhere along one of the streams in the flow-through system 100. In some other cases, there are multiple pumps in the flow-through system 100.

In some other cases, the combining portion 108 is part of the pump 114. For example, the source stream A and the concentrate stream C are combined in a portion of the pump, to generate a combined stream D that is pumped out to the reverse osmosis unit 116. In some other cases, the combining portion 108 is part of the reverse osmosis unit 116. For example, the source stream A and the concentrate stream C are combined in a portion of the reverse osmosis unit 116, to generate a combine stream D, and the stream D within the reverse osmosis unit flows through a separator (e.g., a membrane) to output the filtrate stream B and the concentrate stream C.

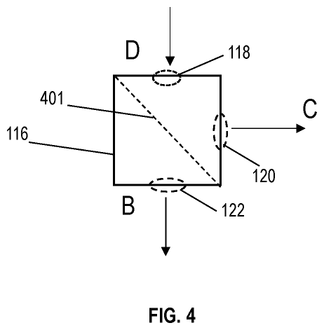

In some cases of the flow-through system 100, the source stream A comprises a first concentration of the radionuclides. Turning briefly to FIG. 4, which shows the reverse osmosis unit 116 in isolation, the reverse osmosis unit 116 comprises a membrane 401 that is configured to separate the combined stream D into the concentrate stream C that comprises a second concentration of the radionuclides that is greater than the first concentration of the radionuclides, and the filtrate stream B that comprises a third concentration of the radionuclides that is less than the second concentration of the radionuclides.

In some cases, the reverse osmosis unit 116 includes multiple membranes 401. In some cases, the reverse osmosis unit 116 is a single stage reverse osmosis system. In some other cases, the reverse osmosis unit 116 is a multi-stage reverse osmosis system that includes multiple membranes to output a filtrate stream B and a concentrate stream C.

Referring back to FIG. 1, in some cases, the flow-through system 100 further includes a first sensor module 138 positioned to measure the concentrate stream C, and the first sensor module 138 is operable to measure a first salinity and a first flow of the concentrated stream C.

In some cases of the flow-through system 100, the first sensor module 138 is positioned downstream from the detection portion output 128.

In some cases, the flow-through system 100 further includes a second sensor module 136 positioned to measure the source stream A, and the second sensor module 136 is operable to measure a second salinity and a second flow of the source stream A.

In some cases, the flow-through system 100 further comprises a third sensor module 140 positioned to be in contact with the filtrate stream B, and the third sensor module 140 operable to measure a third salinity and a third flow of the filtrate stream B.

In some cases, each one of the first sensor module 138, the second sensor module 136 and the third sensor module 140 include a salinity probe and a flowmeter.

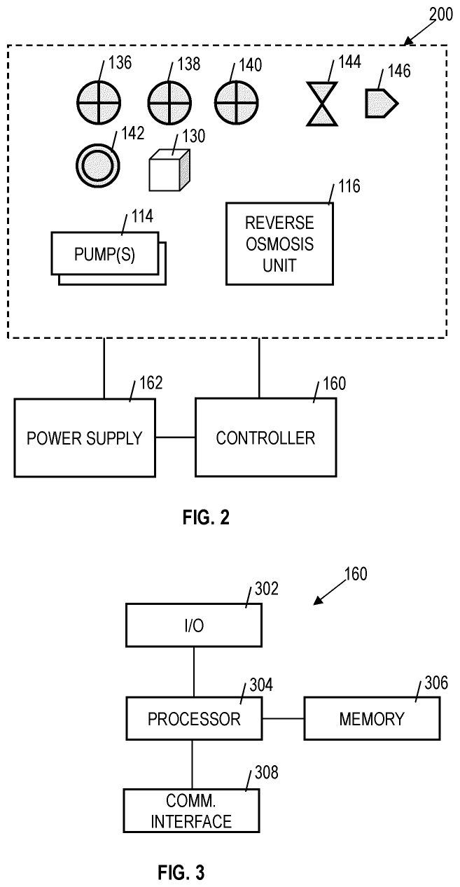

In some cases, as shown in FIG. 1 and as more clearly shown in FIG. 2, a controller 160 interacts with the components of the system. For example, the controller 160 is in data communication with one or more of the components in the set of components 200 including: the first sensor module 138, the second sensor module 136, the third sensor module 140, a pressure gauge 142, a shut-off valve 146, a pressure valve 144, the pump 114 (or multiple pumps), and the spectrometer 130. One more of these components from the set of components 200 can transmit data to or receive data from the controller 160, or both. In some cases, the controller 160 is also in data communication with the reverse osmosis unit 116, whereby the reverse osmosis unit includes data components (e.g., sensors or valves, or both) that transmit or receive data, or both, with the controller 160. In some cases, a power supply 162 provides electrical power to the components, including the controller 160.

In some other cases, one or more of the components from the set of components 200 are not controlled by the controller 160, and instead are manually viewed or controlled. It will be appreciated that the set of components 200 is just an example, and there may be other components in the flow-through system 100.

Referring to FIG. 3, there is illustrated a simplified block diagram of a controller 160 in accordance with at least some embodiments. The controller 160 includes an input/output module 302 (e.g., a touch display screen, audio system, and/or physical buttons), one or more processors 304, one or communication interfaces 308 for receiving and transmitting data, and memory 306. In some cases, the communication interface includes different types of communication modules. In some cases, the communication interface includes a wired module or a wireless communication modules, or both. In some case, the wireless communication module is configured to interact with a cell network, WiFi, BlueTooth, etc.

Referring back to FIG. 1, in some cases, the flow-through system 100 further comprises the controller 160 that is in data communication with at least the first sensor module 138 and the spectrometer 130, and is configured to transmit radioactivity data associated with the concentrated stream C.

In some cases of the flow-through system 100, the controller 160 includes memory 306 that stores executable instructions, and the executable instructions comprise: obtain the measurement of radionuclides in the amount of the concentrated stream within the detection portion, and compute a radioactivity value corresponding to the source stream by at least applying a scaling factor to the measurement of radionuclides corresponding to the concentrated stream.

In some cases of the flow-through system 100, the scaling factor comprises a salinity ratio between the concentrate stream C and the source stream A.

In some cases of the flow-through system 100, the executable instructions further comprise: detecting a steady state associated with at least total dissolved solids (TDS) in the concentrated stream C.

In some cases of the flow-through system 100, the spectrometer 130 is a gamma spectrometer. In some cases, the gamma spectrometer is a cadmium zinc telluride (CZT) semiconductor-based device.

In some cases of the flow-through system 100, the detection portion 124 comprises a vessel defining therein a vessel interior through which the amount of the concentrate stream in the detection portion flows through, and the spectrometer 124 is at least partially positioned within the vessel interior.

In some cases, the flow-through system 100 includes the pressure valve 144 that is operable to interact with the concentrated stream C, including controlling a volume of the concentrated stream C that is recirculated back into the combining portion 108.

In some cases of the flow-through system 100, the pressure valve 144 is positioned downstream from the detection output 128 and upstream from the combining input 110.

In some cases, the flow-through system 100 further comprises an overflow valve 146 that is operable to interact with the concentrate stream C, and an input of the overflow valve 146 is positioned upstream from an input of the pressure valve 011.

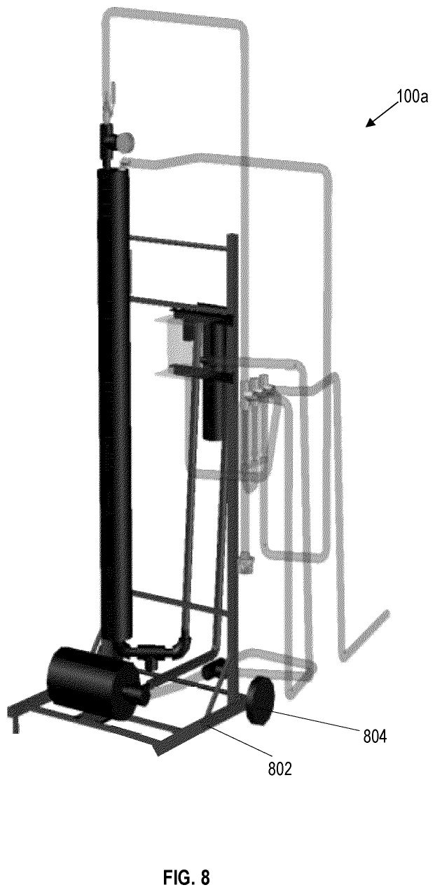

In some cases, the flow-through system 100 further comprises a body, and at least the reverse osmosis unit, the detection portion, the spectrometer, and the combining portion are supported by the body. For example, referring to FIG. 8, a body 802 that supports the components. In some cases, the body 802 is portable and one or more wheels 804 are mounted to the body 802, so that the body 802 may be more easily transported.

In a more general aspect, in some cases the flow-through system 100 is portable. This allows the flow-through system 100 to be moved from one location to another, for example, to monitor different locations in a water body or in a water system.

In some other cases, the flow-through system 100 is fixed to a permanent structure, for example, to monitor radioactivity data in a water system over the long term.

In some cases, as shown in FIG. 1, the flow of the concentrate stream C flows out of the reverse osmosis unit 116 and into and along a concentration loop 132. The concentration loop 132 includes the detection portion 124. The concentrate stream C exits the concentration loop 132 and flows into the combining portion 108, where the concentrate stream C and the source stream A are combined to form a combined stream D.

In some cases, tubing and/or piping is used to fluidically connect the components of the flow-through system 100. For example, an intake tube or a pipe is inserted into the source 102 of aqueous solution to draw out and direct the source stream A from the source 102. Other structures, such as channels, that direct the flow of the streams can be used.

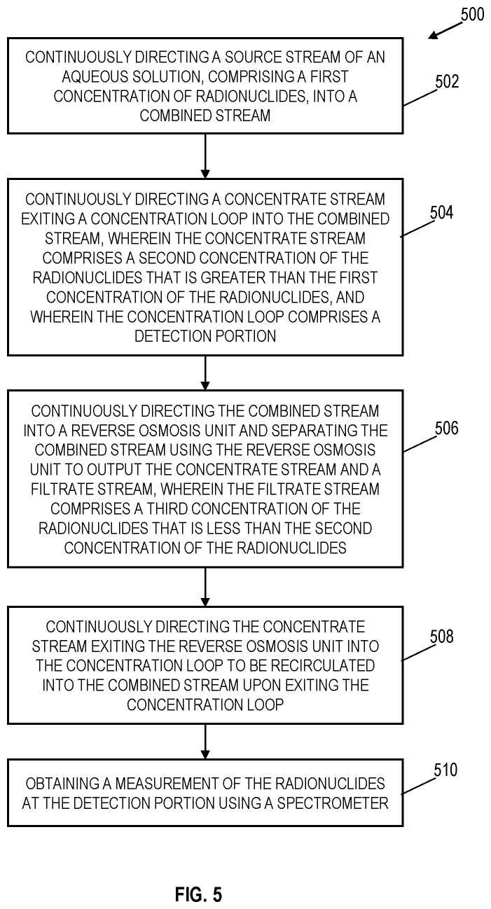

Referring now to FIG. 5, an example flow-through method 500 for measuring radionuclides in aqueous solution is provided.

At operation 502, the method includes continuously directing a source stream A of an aqueous solution, comprising a first concentration of radionuclides, into a combined stream D.

At operation 504, the method includes continuously directing a concentrate stream C exiting a concentration loop 132 into the combined stream D, wherein the concentrate stream C comprises a second concentration of the radionuclides that is greater than the first concentration of the radionuclides, and wherein the concentration loop 132 comprises a detection portion 124.

At operation 506, the method includes continuously directing the combined stream D into a reverse osmosis unit 116 and separating the combined stream D using the reverse osmosis unit 116 to output the concentrate stream C and a filtrate stream B, wherein the filtrate stream B comprises a third concentration of the radionuclides that is less than the second concentration of the radionuclides.

At operation 508, the method includes continuously directing the concentrate stream C exiting the reverse osmosis unit 116 into the concentration loop 132 to be recirculated into the combined stream D upon exiting the concentration loop 132.

At operation 510, the method includes obtaining a measurement of the radionuclides at the detection portion 124 using a spectrometer 130.

In some cases, the spectrometer 130 repeatedly measures the radionuclides at the detection portion 124 in near real-time.

In some cases, the method further includes obtaining a salinity ratio representing a relationship between a first salinity of the concentrate stream C and a second salinity of the source stream A, and computing, using the controller 160, a value for the first concentration of radionuclides present in the source stream A using at least the salinity ratio and the measurement of radionuclides at the detection portion 124.

In some cases, the first sensor module 138 is positioned in the concentration loop 132 and repeatedly measures a first salinity and a first flow of the concentrate stream C. The second sensor module 136 is positioned upstream from the reverse osmosis unit 116 and repeatedly measures a second salinity and a second flow of the source stream A prior to entering the reverse osmosis unit 116.

In some cases, the controller 160 obtains a value of the first salinity from the first sensor module 138 and a value of the second salinity from the second sensor module. The controller 160 computes a salinity ratio using the value of the first salinity and the value of the second salinity. The controller 160 then computes a value for the first concentration of radionuclides present in the source stream A using at least the salinity ratio and the measurement of radionuclides at the detection portion 124.

In some cases, the third sensor module 140 is positioned downstream from the reverse osmosis unit 116 and repeatedly measures a third salinity and a third flow of the filtrate stream B outputted by the reverse osmosis unit 116.

In some cases, the third concentration of the radionuclides in the filtrate stream B is less than the first concentration of the radionuclides in the source stream A.

In some cases, the third concentration of the radionuclides in the filtrate stream B is about equal to the first concentration of the radionuclides in the source stream A.

In some cases, the obtaining the measurement of the radionuclides at the detection portion 124 occurs while the concentrate stream C is flowing through the detection portion 124.

In some cases, the spectrometer 130 is a gamma spectrometer. In some cases, the gamma spectrometer is a cadmium zinc telluride (CZT) semiconductor-based device.

In some cases, the spectrometer 130 is at least partially positioned within the detection portion 124.

In some cases, the method further includes detecting that a steady state of total dissolved solids in the concentration loop 132 is achieved.

In some cases, the method further includes transmitting radioactivity data using a communication interface 308, wherein the radioactivity data comprises the measurement of the radionuclides, or the radioactivity data is derived from the measurement of the radionuclides.

In some cases, the method further includes outputting radioactivity data via a user I/O device 302, wherein the radioactivity data comprises the measurement of the radionuclides, or the radioactivity data is derived from the measurement of the radionuclides.

In some cases of the flow-through method, the radioactivity data comprises an alert indicating that the measurement of the radionuclides is above a threshold value.

In some cases, the method further includes: performing the method 500 at a first time and obtaining a first instance of the measurement of the radionuclides, corresponding to the first time, and computing a first instance value of the first concentration of radionuclides, corresponding to the first time, using at least the first instance of the measurement of the radionuclides. The method further includes, repeating the method 500 at a second time and obtaining a second instance of the measurement of the radionuclides, corresponding to the second time, wherein the second time occurs within a predetermined time period after the first time. The method further includes computing a second instance value of the first concentration of radionuclides, corresponding to the second time, using at least the second instance of the measurement of the radionuclides.

The method continues with comparing the first instance value of the first concentration of radionuclides, to the second instance value of the first concentration of radionuclides. The method further includes triggering an alert after detecting that the second instance value of the first concentration of radionuclides, has increased from the first instance value of the first concentration of radionuclides, by at least a given threshold value.

In some other cases, the method further includes performing the method 500 at a first time and obtaining a first instance of the measurement of the radionuclides, corresponding to the first time. Then, computing a first instance value of the first concentration of radionuclides, corresponding to the first time, using at least the first instance of the measurement of the radionuclides. The method further includes triggering an alert after detecting that the first instance value of the first concentration of radionuclides is greater than a given threshold value.

In some cases, the method further comprises discarding the filtrate stream B outputted by the reverse osmosis unit 116.

In some cases, the method further includes controlling an amount of the concentrate stream C being recirculated in the concentration loop 132 using a pressure valve 144 that is in fluidic communication with the concentration loop 132.

In some cases, an initial amount of the source stream A from the source 102 with a concentration of radionuclides cₒ is provided by the pump 114 to the reverse osmosis unit 116 comprising a reverse osmosis membrane 401. In other words, the term co refers to the first concentration of radionuclides in the initial amount of the stream A.

At least a portion of the aqueous solution from the source stream A permeates through the reverse osmosis membrane 401 to form the filtrate stream B with a concentration of radionuclides less than co and a concentrate stream C with a concentration of the radionuclides greater than co is retained in the system.

The concentrate stream C is passed through the detection portion 124 prior to mixing with the source stream A to form combined stream D wherein the flow of sample stream A is determined by the pressure setting of the system.

Combined stream D is returned to the reverse osmosis unit 116 to further form a filtrate stream B and a concentrate stream C with an increased concentration of the radionuclides.

The concentrate stream C is continuously recirculated by the pump 114 through the concentration loop 132 to mix with source stream A to again form a combined stream D which is continuously supplied to the reverse osmosis unit 116. In some cases, this process is continuous. Overtime, a steady-state may be achieved in the flow-through system 100 which, in some cases, is measured by the total dissolved solids in the concentration loop 132.

The detection portion 124 includes the gamma spectrometer and is configured to allow the detection of the radionuclides.

Using the flow-through system or the flow-through method, or both, the radionuclides in the aqueous solution are concentrated via reverse osmosis by recirculating the concentrate stream C back into the combined stream D. For example, this recirculation is represented by the concentration loop 132. The concentrate stream C flows through the detection portion 124. In some cases, it is a CZT gamma type spectrometer in the center of the detection portion 124 that continuously tracks the radionuclide content with radionuclide detection limits in the order of 1-5 Bq/L or better with extended counting times.

The spectrometer 130 measures a physical count rate of a species, Ci, which is related to the activity of that species in the water reservoir, Af,i, by the gamma energy-dependent detection efficiency, ϵi:

$${\dot{C}}_{i} = {ϵ}_{i} {A}_{f , i}$$

Reverse osmosis systems take feed water with a given background concentration cₒ, and create a filtrate stream with a lower concentration, c_{F}, and a concentrate stream with a higher concentration, c_{c}. The efficiency is given by the ratios R_{F}=c_{F}/cₒ and R_{c}=c_{c}/cₒ. However, a larger degree of concentration can be achieved by recirculating the concentrate stream back through the system in a loop, mixing it with a certain amount of sampling water, that feeds in with a flow rate of Fₛ and that has an initial concentration of cₛ. As such, the concentration in the loop will increase overtime according to:

$${c}_{c} ( t ) = {c}_{s} \frac{{R}_{c}}{{R}_{F}} ( 1 - {e}_{F}^{- \frac{{R}_{F}}{r} \text{?}} )$$

$$\text{?} \text{indicates text missing or illegible when filed}$$

In this expression, τ=V/Fₛ is the residence time of water in the loop, given the volume of the loop and sampling flow rate. It will take time for the dissolved materials to build up in concentration, but this is achievable in a reasonable amount of time as long as the volume in the loop is minimized or the sampling rate is increased. The steady concentration that the system will approach depends on the efficiency of the system, although it must also be noted that the efficiencies R_{c} and R_{F} can both also vary depending on the solute concentrations in the system. Most of the concentration will be driven by naturally occurring dissolved solids, and any radioactive substances will mainly follow those.

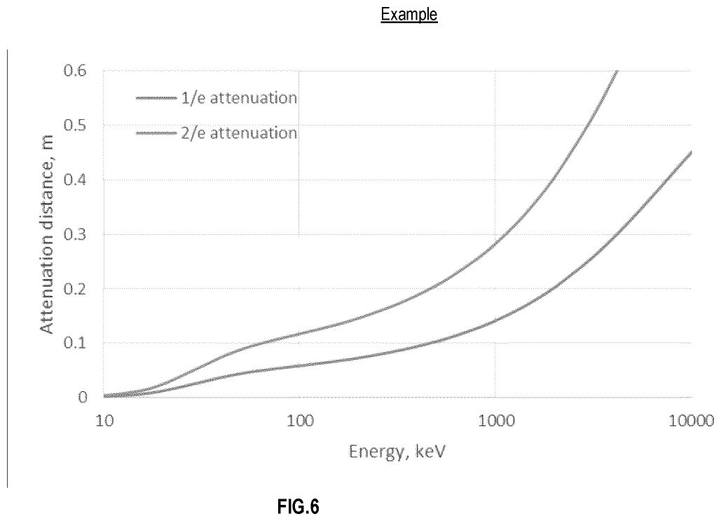

It is desired to have a large enough detection portion 124, i.e., a detection portion 124 that holds at least a predetermined amount of aqueous solution, in order to have at least a certain amount of radionuclides within the detection portion that emit at least a certain amount of radioactivity in view of the gamma spectrometer, since A_{f,i}=V_{f}c_{c}. However, there is a point of diminishing return since the aqueous solution (or water) will self-shield gamma rays, and in particular those of low energy. This is demonstrated in FIG. 6, which gives the approximate gamma attenuation distance with the detector in the center of a water reservoir (e.g., a detection portion) with a uniform concentration. The most effective size for gamma rays with energies between 100 keV and 1000 keV would be to have a radius of around 5 to 15 cm.

### Concentration Loop

A schematic of the concentration loop 132 is given in FIG. 1, according to an example embodiment. In some cases, the source stream A from a contaminated source 102 flows into the flow-through system 100 and drawn in with a pump 114. The same pump drives the overall recirculation through the flow-through system, and the pressure that drives the reverse osmosis process. The clean filtrate stream B that is discharged from the osmosis membrane is discarded. The concentrate stream C first circulates to the detection portion, prior to flowing back to mix with the source stream A, to form a combined stream D. In some cases, the amount of the concentrate stream C that is recirculated is controlled by the setting a pressure valve 144. In some cases, this also determines how much of the filtrate to discard. There is also an overflow valve 146 of the concentration loop 132 that helps with initially filling the system with the aqueous solution (or water).

### Detection Portion

It is desired to have a large enough detection portion 124 in order to have enough radioactivity in view of the gamma spectrometer, since A_{f,i}=V_{f}c_{c}. However, there is a point of diminishing return since water will self-shield gamma rays, and in particular those of low energy. This is demonstrated in FIG. 6, which gives the approximate gamma attenuation distance with the spectrometer in the center of a water reservoir with a uniform concentration. In some cases, an effective size for gamma rays with energies between 100 keV and 1000 keV would be to have a radius of around 5 to 15 cm.

### Gamma Ray Spectrometer

One example of a suitable gamma spectrometer is a cadmium zinc telluride (CZT) semiconductor-based device. In some cases, it has better spectral resolution and is more compact than sodium iodide scintillator detectors, but can operate at room temperature, without the need for cryogenics as is the case for germanium-based detectors. The CZT detectors are usually very small in size, with crystals on the order of 1 cm³. In the prototype, a Kromek GR1 CZT detector was used specifically. The Kromek MultiSpect Analysis software is employed to capture and record the gamma spectrometry measurements.

### Calculated Counting Efficiency

This section describes an example method to determine the efficiency of the aquatic gamma detector using a geant4 based simulation. Results from simulations for a number of isotopes are also shown. In some cases, including in the section, the spectrometer 130 is referred to as a detector.

To simulate the gammas within the detector the RAT1software was used. The version of geant used by RAT was 4.10.04.p02. The physics list used was the Shielding physics list.

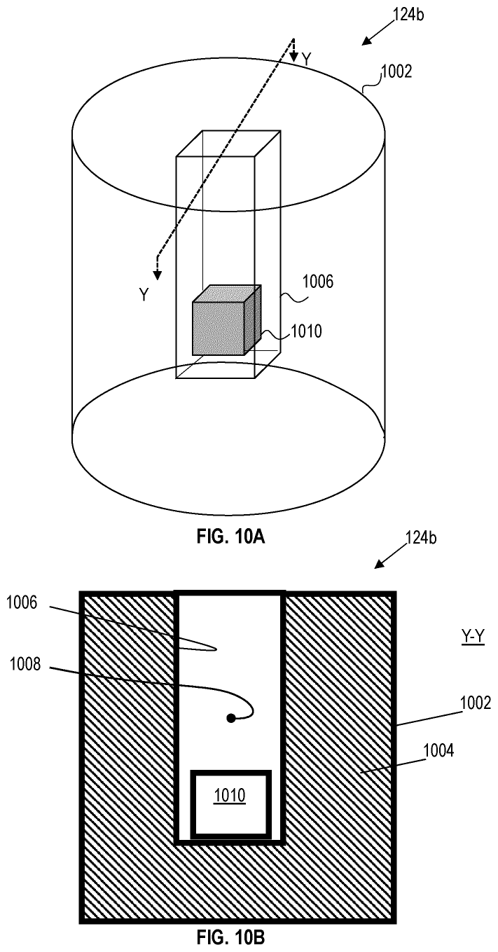

A simple geometry of the detection portion 124b was constructed based on the provided CAD drawings. As per FIGS. 10A and 10B, in some cases the geometry includes of a cylindrical container 1002 that has interior that holds a volume of aqueous solution 1004, in which is a holder body 1006. In some cases, the holder body 1006 is a hollow polycarbonate box. The interior 1008 defined within the holder body 1006 is filled with air and holds a small CdZnTe crystal 1010 for sensing radioactivity.

The parameters used in the simulation for the geometry shown in FIGS. 10A and 10B are listed in Table 1.

TABLE 1
Simulation geometry parameters

| Volume | Dimensions | Material |
|---|---|---|
| Water | Cylinder: Radius 2.25″Length: 5″ | Water |
| Detector | Width: 1.25 × 1.25″ Length: 3″ | Polycarbonate, density 1.2 g/cm³ |
| Holder | Wall thickness: 0.125″ | Composition by mass: (H: 0.06, C: |
| | | 0.76, O: 0.19) |
| Detector | 1 cm³ at the bottom of the | CdZnTe Crystal, density 5.9 g/cm³ |
| Crystal | holder | Composition by mass (Cd: 0.39, Zn: |
| | | 0.06, Te: 0.55) |

For each of the selected isotopes 1 million decays were simulated in the water volume. Gammas produced from the de-excitation of the daughter nucleus were then tracked to see if they deposited energy into the crystal. The fraction of the decays where at least one de-excitation gamma from the decay enters the crystal gives the geometric efficiency. The overall efficiency is given by the fraction of decays which produce a de-excitation gamma which then goes onto deposit energy within the detector crystal. The uncertainty on these values is the 10 binomial uncertainty.

The seven simulated isotopes can be seen in Table 2. The efficiency of the detector is dominated by geometric effects. Of all the gammas produced only 0.7% enter the detector crystal. Isotopes which produce more gammas during their decays will have a higher geometric efficiency per decay due to the increased number of gamma rays emitted. The mean number of gammas per decay and the two efficiencies and their uncertainties are shown in the table below. Decays which produce more gammas tend to have a higher geometric efficiency. The overall efficiency is also altered by other energy dependent effects.

TABLE 2
Simulation results

| | | | | Overall | |
|---|---|---|---|---|---|
| | Mean | | | Efficiency | |
| | number of | Geometric | Geometric | (Per Decay) | Overall |
| | Gammas | Efficiency | Efficiency | (Geometric + | Efficiency |
| Isotope | per Decay | (Per Decay) | Error | Detection) | Error |
| Cs134 | 2.23767 | 0.01532 | 0.000123 | 0.004987 | 7.04E−05 |
| Te132 | 1.87808 | 0.010984 | 0.000104 | 0.008638 | 9.25E−05 |
| Ru103 | 1.11898 | 0.007147 | 8.42E−05 | 0.002806 | 5.29E−05 |
| I131 | 1.0646 | 0.007397 | 8.57E−05 | 0.003668 | 6.05E−05 |
| La140 | 2.16459 | 0.01436 | 0.000119 | 0.004404 | 6.62E−05 |
| Ce144 | 0.233478 | 0.001597 | 3.99E−05 | 0.001472 | 3.83E−05 |
| Cs137 | 0.931168 | 0.00619 | 7.84E−05 | 0.002333 | 4.82E−05 |

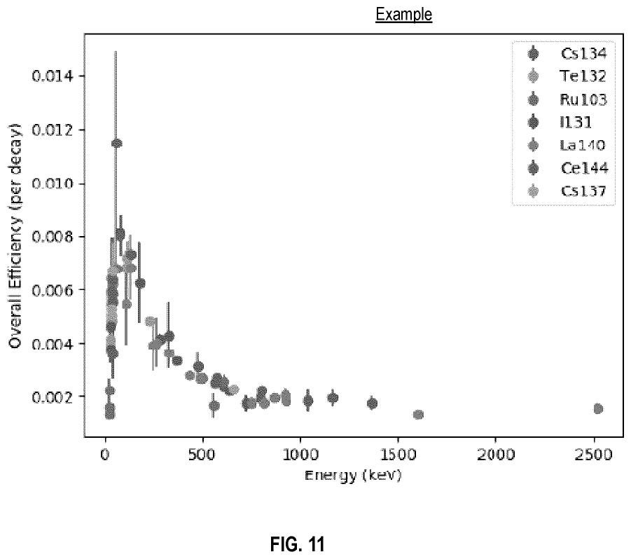

The efficiency that once the gamma enters the detector it deposits energy within it depends on the energy of the gamma. As the decays often produce several distinct gamma lines the gammas from each isotope were grouped in energy and their efficiencies as a function of energy were plotted as seen in FIG. 11. The detector efficiency peaks at around 100 keV. Below this the efficiency drops off sharply. Above this the efficiency gradually decreases up to 750 keV.

### Prototype—Example Embodiment

The prototype is an example embodiment and uses the Mini Hobby RO as its reverse osmosis unit to which a recirculation loop is added. That is, the system base is the Mini Hobby RO which uses reverse osmosis to produce a concentrate from the water from the intake. A concentration loop was added to revert the concentrate back into the system being supplemented by the intake for the excess required. A reservoir, acting as the detection portion, has also been added to the concentration loop to have an area for the gamma spectrometer to monitor for radionuclides.

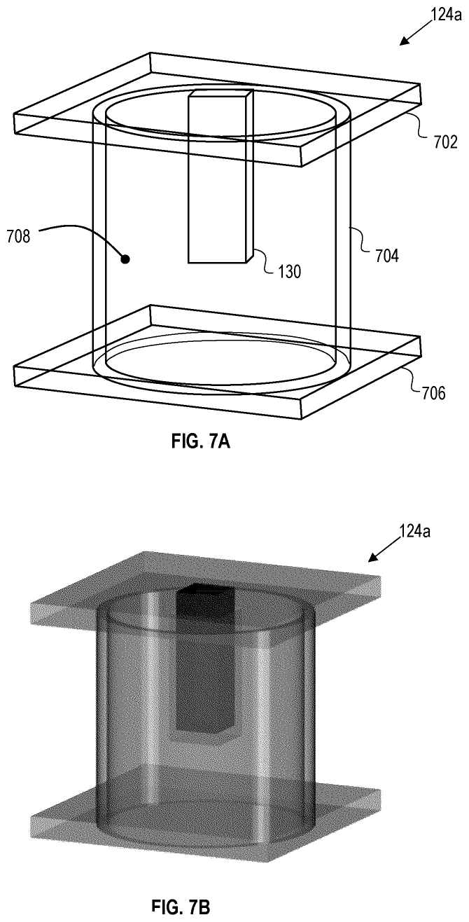

Referring also to FIGS. 7A and 7B, the detection portion 124a used for gamma counting prototype was constructed out of polycarbonate. It includes a cylinder body 704 with about a 12.7 cm length and about a 5.715 cm internal diameter. The gamma spectrometer 130 is slotted into a section in the center of the cylinder body 704, which was also constructed out of polycarbonate, so that it will not have to come into direct contact with the aqueous solution (or water). In the example prototype, the volume of water that the detection portion can contain is 1.226 L. In the field application, the overall steady concentration ratio (R_{c}/R_{F}) is about 15, meaning that the combination of the reverse osmosis unit and the concentration loop is able to concentrate dissolved materials in the concentration loop by about 15 times the concentration in the environmental sample.

It will be appreciated that the detection portion 124 can have other shapes and configurations that are different from the examples shown in the figures provided.

### Field Testing of Prototype

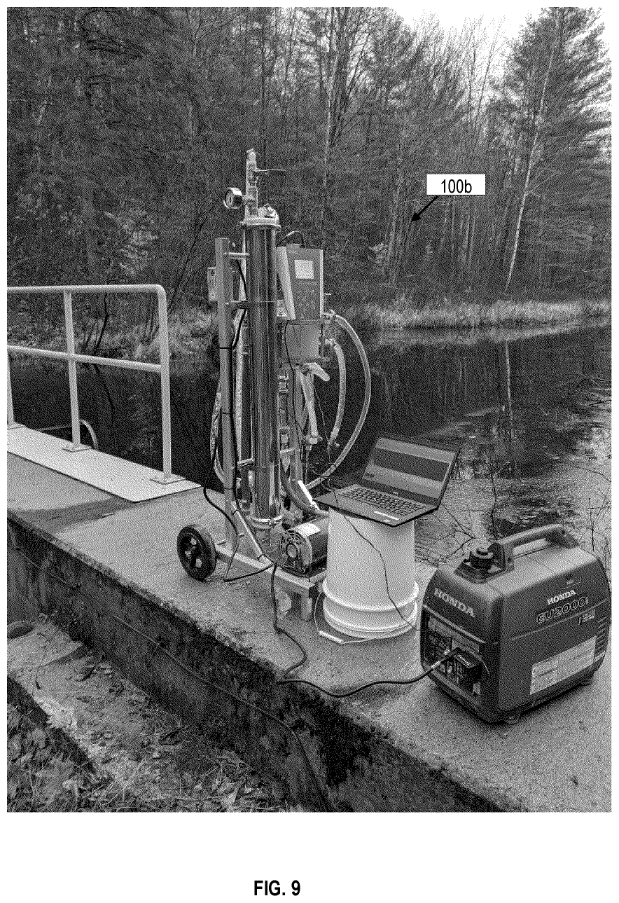

A field test on the prototype 100b of the flow-through system 100b was conducted. A photo of the prototype 100b during the field trial is shown in FIG. 9. A portable generator (which is a type of power supply) was used to supply electricity. The intake tube with filter was placed approximately 30 cm into the water to avoid the silt from clogging the sampler. The prototype 100b was run without applying differential pressure to get a baseline concentration at first, which was found to be about 80 mg/L based on the salinity measurements, and also to fill the system with water. When the detection portion was filled the concentration loop was opened, recirculating the concentrated water back into the system. Upon initiating the recirculation mode, the system could build up in concentration. In the field test example, a peak concentration of about 1200 mg/L was achieved after about 50 min of operation.

No radionuclides were detected above background levels, but this was as expected, since the water did not contain any dissolved gamma emitting substances.

It is possible to estimate the projected performance of the system, however. In real-world applications, the overall steady concentration ratio (R_{c}/R_{F}) is about 15, meaning that the reverse osmosis and recirculation system is able to concentrate dissolved materials in the loop by about 15 times the concentration in the environmental sample. Given the volume of the water reservoir (e.g., the detection portion) and the fact that counting efficiency ranges from 0.002 to 0.008 for most radionuclides, it is expected that the detection limit of the system (the value of cₛ that would yield a count rate, Cᵢ, of about 10 cpm in the gamma spectrometer) is around 1.1 to 4.5 Bq/L. Higher concentrations would, of course, lead to higher count rates. It would also be reasonable to extend the counting time to achieve even lower detection limits.

An objective of the prototype testing was to develop an exemplary aquatic radionuclide sampler, using at least some of the teachings described herein that was able to be deployed in the field. The target for this phase of the testing was to be able to detect radionuclide concentration at least in the range required to make decisions about drinking water OILs (range of 100 to 1000 Bq/L). The current prototype design used a reverse osmosis system and concentration loop to raise the concentration of dissolved radionuclides from sampling. The system also contains a gamma spectrometer that can collect data live, in the field. The current design accomplishes the stated goals, as the testing determined that the prototype system is able to achieve detection limits on the order of 1-5 Bq/L, depending on the gamma energy of the radionuclide of interest.

### Other Example Aspects

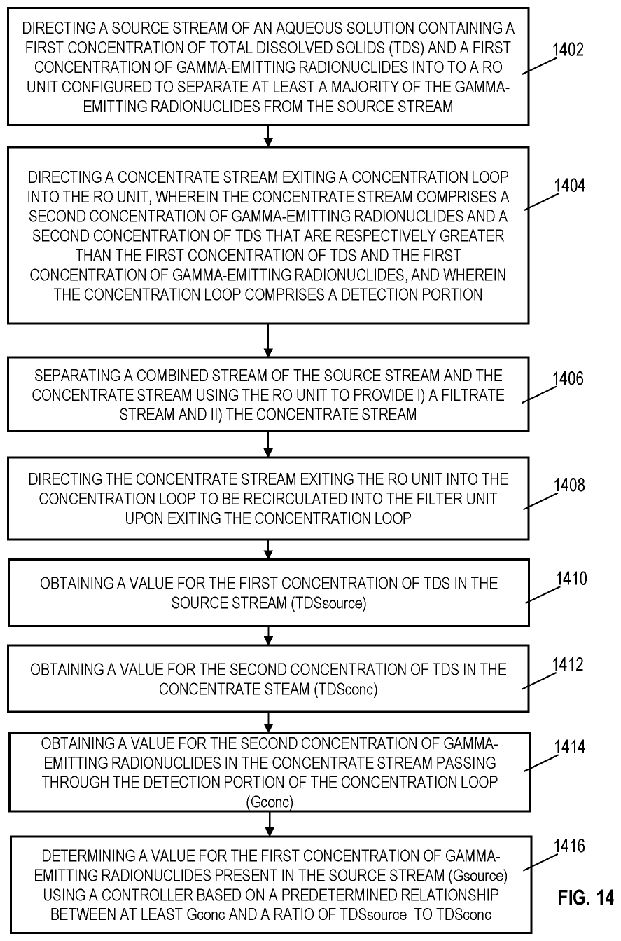

A flow-though method is provided for determining a concentration of gamma-emitting radionuclides in an aqueous solution. Referring to FIG. 14, the method includes the below operations:

Operation 1402: directing a source stream of an aqueous solution containing a first concentration of total dissolved solids (TDS) and a first concentration of gamma-emitting radionuclides into to a reverse osmosis unit configured to separate at least a majority of the gamma-emitting radionuclides from the source stream.

Operation 1404: directing a concentrate stream exiting a concentration loop into the reverse osmosis unit, wherein the concentrated stream comprises a second concentration of gamma-emitting radionuclides and a second concentration of TDS that are respectively greater than the first concentration of TDS and the first concentration of gamma-emitting radionuclides, and wherein the concentration loop comprises a detection portion.

Operation 1406: separating a combined stream of the source stream and the concentrate stream using the reverse osmosis unit to provide i) a filtrate stream and ii) the concentrate stream;

Operation 1408: directing the concentrate stream exiting the reverse osmosis unit into the concentration loop to be recirculated into the reverse osmosis unit upon exiting the concentration loop.

Operation 1410: obtaining a value for the first concentration of TDS in the source stream, TDS_{source}.

Operation 1412: obtaining a value for the second concentration of TDS in the concentrate steam, TDS_{conc}.

Operation 1414: obtaining a value for the second concentration of gamma-emitting radionuclides in the concentrate stream passing through the detection portion of the concentration loop, G_{conc}.

Operation 1416: determining a value for the first concentration of gamma-emitting radionuclides present in the source stream, G_{source}, using a controller based on a predetermined relationship between at least G_{conc} and a ratio of TDS_{source} to TDS_{conc}.

In some cases, the reverse osmosis unit comprises a membrane configured to separate the gamma-emitting radionuclides from the source stream and retain them in the concentrated stream.

In some cases, the obtaining the value for the second concentration of gamma-emitting radionuclides in the concentrate stream passing through the detection portion comprises measuring the second concentration of gamma-emitting radionuclides using a gamma spectrometer configured to measure the second concentration of gamma-emitting radionuclides in the concentrate stream while the concentrate stream is flowing through the detection portion.

In some cases, the concentrate stream comprises a majority of the gamma-emitting radionuclides entering the reverse osmosis unit.

In some cases, the method further comprises obtaining a value of a third concentration of TDS in the filtrate stream, and wherein the value of the third concentration of TDS is substantially equal to the value of the first concentration of TDS.

In some cases, the method further comprises obtaining a value of a third concentration of TDS in the filtrate stream, and wherein a difference between the value of the third concentration of TDS and the value of the first concentration of TDS is lower than a predetermined difference threshold.

In some cases, the predetermined relationship, G_{source} is proportional to (TDS_{source}/TDS_{conc})*G_{conc}. In some cases, TDS_{source} is obtained by measuring the TDS in the source stream before it enters the reverse osmosis unit. In some cases, TDS_{conc} is obtained by measuring the TDS in the concentrate stream flowing in the concentration loop. In some cases, TDS_{conc} is obtained by measuring the TDS in the concentrate stream flowing in the concentration loop and before mixing with the source stream.

In some cases, the concentrate stream and the source stream are mixed together before entering the reverse osmosis unit.

In some cases, the method further includes detecting that a steady state of TDS in the concentrate stream has been achieved, and, after detecting the steady state, proceeding with the obtaining of TDS_{source}, TDS_{conc}, and G_{conc}, and the determining of G_{source}.

In some cases, detecting that the steady state is achieved comprises: computing at different instances in time a plurality of values of TDS_{conc} corresponding to the different instances in time, and detecting that consecutive ones of the plurality of values of TDS_{conc}, within a pre-defined time period, are within a pre-defined variance compared to each other.

In some cases, detecting that the steady state is achieved comprises repeatedly measuring a TDS in the concentration loop over time and detecting that a plurality of consecutive measurements of the TDS in the flow loop are within a pre-defined variance of each other.

In some cases, a mass flow rate of the source stream is equal to a mass flow rate of the filtrate stream.

In some cases, the mass flow rate of the concentrate stream is less than the mass flow rate of the source stream.

Various systems or processes have been described to provide examples of embodiments of the claimed subject matter. No such example embodiment described limits any claim and any claim may cover processes or systems that differ from those described. The claims are not limited to systems or processes having all the features of any one system or process described above or to features common to multiple or all the systems or processes described above. It is possible that a system or process described above is not an embodiment of any exclusive right granted by issuance of this patent application. Any subject matter described above and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

For simplicity and clarity of illustration, reference numerals may be repeated among the figures to indicate corresponding or analogous elements. In addition, numerous specific details are set forth to provide a thorough understanding of the subject matter described herein. However, it will be understood by those of ordinary skill in the art that the subject matter described herein may be practiced without these specific details. In other instances, well-known methods, procedures, and components have not been described in detail so as not to obscure the subject matter described herein.

As used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

Terms of degree such as “substantially”, “about”, and “approximately” as used herein mean a reasonable amount of deviation of the modified term such that the result is not significantly changed. These terms of degree may also be construed as including a deviation of the modified term if this deviation would not negate the meaning of the term it modifies.

Any recitation of numerical ranges by endpoints herein includes all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the result is not significantly changed.

Some elements herein may be identified by a part number, which is composed of a base number followed by an alphabetical or subscript-numerical suffix (e.g., 124a, or 124b). All elements with a common base number may be referred to collectively or generically using the base number without a suffix (e.g., 124).

The systems and methods described herein may be implemented as a combination of hardware or software. In some cases, the systems and methods described herein may be implemented, at least in part, by using one or more computer programs, executing on one or more programmable devices including at least one processing element, and a data storage element (including volatile and non-volatile memory and/or storage elements). These systems may also have at least one input device (e.g. a pushbutton keyboard, mouse, a touchscreen, and the like), and at least one output device (e.g. a display screen, a printer, a wireless radio, and the like) depending on the nature of the device.

Some elements that are used to implement at least part of the systems, methods, and devices described herein may be implemented via software that is written in a high-level procedural language such as object-oriented programming language. Accordingly, the program code may be written in any suitable programming language such as Python or Java, for example. Alternatively, or in addition thereto, some of these elements implemented via software may be written in assembly language, machine language or firmware as needed. In either case, the language may be a compiled or interpreted language.

At least some of these software programs may be stored on a storage media (e.g., a computer readable medium such as, but not limited to, read-only memory, magnetic disk, optical disc) or a device that is readable by a general or special purpose programmable device. The software program code, when read by the programmable device, configures the programmable device to operate in a new, specific, and predefined manner to perform at least one of the methods described herein.

Furthermore, at least some of the programs associated with the systems and methods described herein may be capable of being distributed in a computer program product including a computer readable medium that bears computer usable instructions for one or more processors. The medium may be provided in various forms, including non-transitory forms such as, but not limited to, one or more diskettes, compact disks, tapes, chips, and magnetic and electronic storage. Alternatively, the medium may be transitory in nature such as, but not limited to, wire-line transmissions, satellite transmissions, internet transmissions (e.g., downloads), media, digital and analog signals, and the like. The computer usable instructions may also be in various formats, including compiled and non-compiled code.

While the above description provides examples of one or more processes or systems, it will be appreciated that other processes or systems may be within the scope of the accompanying claims.

It will also be appreciated that different example aspects and features in different cases and embodiment described herein, can be combined with each other in the context of measuring radionuclides in aqueous solution, although the combination may not be explicitly stated.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be revisited.

What is claimed is:

1. A flow-though method for measuring radionuclides in an aqueous solution, the method comprising:
continuously directing a source stream of an aqueous solution, comprising a first concentration of radionuclides, into a combined stream;
continuously directing a concentrate stream exiting a concentration loop into the combined stream, wherein the concentrate stream comprises a second concentration of the radionuclides that is greater than the first concentration of the radionuclides, and wherein the concentration loop comprises a detection portion;
continuously directing the combined stream into a reverse osmosis unit and separating the combined stream using the reverse osmosis unit to output the concentrate stream and a filtrate stream, wherein the filtrate stream comprises a third concentration of the radionuclides that is less than the second concentration of the radionuclides;
continuously directing the concentrate stream exiting the reverse osmosis unit into the concentration loop to be recirculated into the combined stream upon exiting the concentration loop; and
obtaining a measurement of the radionuclides at the detection portion using a spectrometer.

2. The flow-through method of claim 1, wherein the spectrometer repeatedly measures the radionuclides at the detection portion in near real-time.

3. The flow-through method of claim 1 or claim 2, further comprising: obtaining a salinity ratio representing a relationship between a first salinity of the concentrate stream and a second salinity of the source stream; and computing, using a controller, a value for the first concentration of radionuclides present in the source stream using at least the salinity ratio and the measurement of radionuclides at the detection portion.

4. The method of claim 1 or claim 2, wherein a first sensor module is positioned in the concentration loop and repeatedly measures a first salinity and a first flow of the concentrate stream; and a second sensor module is positioned upstream from the reverse osmosis unit and repeatedly measures a second salinity and a second flow of the source stream prior to entering the reverse osmosis unit.

5. The method of claim 4, further comprising:
a controller obtaining a value of the first salinity from the first sensor module and a value of the second salinity from the second sensor module;
the controller computing a salinity ratio using the value of the first salinity and the value of the second salinity;
the controller computing a value for the first concentration of radionuclides present in the source stream using at least the salinity ratio and the measurement of radionuclides at the detection portion.

6. The method of claim 4, wherein a third sensor module is positioned downstream from the reverse osmosis unit and repeatedly measures a third salinity and a third flow of the filtrate stream outputted by the reverse osmosis unit.

7. The flow-through method of any one of claims 1 to 6, wherein the third concentration of the radionuclides in the filtrate stream is less than the first concentration of the radionuclides in the source stream.

8. The flow-through method of any one of claims 1 to 6, wherein the third concentration of the radionuclides in the filtrate stream is about equal to the first concentration of the radionuclides in the source stream.

9. The method of any one of claims 1 to 8, wherein the obtaining the measurement of the radionuclides at the detection portion occurs while the concentrate stream is flowing through the detection portion.

10. The method of any one of claims 1 to 9, wherein the spectrometer is a gamma spectrometer.

11. The method of claim 10, wherein the gamma spectrometer is a cadmium zinc telluride (CZT) semiconductor-based device.

12. The method of any one of claims 1 to 11, wherein the spectrometer is at least partially positioned within the detection portion.

13. The method of any one of claims 1 to 12, further comprising detecting that a steady state of total dissolved solids in the concentration loop is achieved.

14. The method of any one of claims 1 to 13, further comprising transmitting radioactivity data using a communication interface, wherein the radioactivity data comprises the measurement of the radionuclides, or the radioactivity data is derived from the measurement of the radionuclides.

15. The method of any one of claims 1 to 13, further comprising outputting radioactivity data via a user I/O device, wherein the radioactivity data comprises the measurement of the radionuclides, or the radioactivity data is derived from the measurement of the radionuclides.

16. The method of claim 14 or claim 15, wherein the radioactivity data comprises an alert indicating that the measurement of the radionuclides is above a threshold value.

17. The method of any one of claims 1 to 16, further comprising:
performing the method of claim 1 at a first time and obtaining a first instance of the measurement of the radionuclides, corresponding to the first time;
computing a first instance value of the first concentration of radionuclides, corresponding to the first time, using at least the first instance of the measurement of the radionuclides;
repeating the method of claim 1 at a second time and obtaining a second instance of the measurement of the radionuclides, corresponding to the second time, wherein the second time occurs within a predetermined time period after the first time;
computing a second instance value of the first concentration of radionuclides, corresponding to the second time, using at least the second instance of the measurement of the radionuclides;
comparing the first instance value of the first concentration of radionuclides, to the second instance value of the first concentration of radionuclides; and
triggering an alert after detecting that the second instance value of the first concentration of radionuclides has increased from the first instance value of the first concentration of radionuclides by at least a given threshold value.

18. The method of any one of claims 1 to 16, further comprising:
performing the method of claim 1 at a first time and obtaining a first instance of the measurement of the radionuclides, corresponding to the first time;
computing a first instance value of the first concentration of radionuclides, corresponding to the first time, using at least the first instance of the measurement of the radionuclides;
triggering an alert after detecting that the first instance value of the first concentration of radionuclides is greater than a given threshold value.

19. The method of any one of claims 1 to 18, further comprising discarding the filtrate stream outputted by the reverse osmosis unit.

20. The method of any one of claims 1 to 19, further comprising controlling an amount of the concentrate stream being recirculated in the concentration loop using a pressure valve that is in fluidic communication with the concentration loop.

21. A flow-through system for monitoring radionuclides in aqueous solution, the flow-through system comprising:
a flow-through system input configured to receive a source stream of an aqueous solution;
a reverse osmosis unit comprising a reverse osmosis input, a reverse osmosis output, and a concentrate output;
a detection portion comprising a detection portion input and a detecting portion output;
a spectrometer positioned at the detection portion;
a combining portion comprising a combining input and a combining output; and
a flow-through system output;
wherein:
the combining input is in fluidic communication with the flow-through system input and the detection portion output, and is configured to receive the source stream from the flow-through system input and a concentrate stream outputted by the detecting portion output;
the combining output is configured to output a combined stream, which comprises the source stream and the concentrate stream outputted by the detecting portion output;
the reverse osmosis input is in fluidic communication with the combining output and is configured to receive the combined stream;
the reverse osmosis unit is configured to separate the combined stream into the concentrate stream and a filtrate stream, output the concentrate stream via the concentrate output, and output the filtrate stream via the reverse osmosis output;
the concentrate output is in fluidic communication with the detection portion input and the detection portion input is configured to receive the concentrate stream outputted by the concentrate output;
the detection portion output is configured to recirculate the concentrate stream into the combined stream;
the spectrometer is operable to obtain a measurement of radionuclides in an amount of the concentrate stream within the detection portion; and
the flow-through system output is in fluidic communication with the reverse osmosis output and is configured to output the filtrate stream.

22. The flow-through system of claim 21, further comprising a pump operable to: continuously flow the source stream through the flow-through system input, continuously flow the combined stream into the reverse osmosis input, continuously flow the concentrate stream through the detection portion and recirculate the concentrate stream back into the combined portion, and continuously flow the filtrate stream out the flow-through system output.

23. The flow-through system of claim 22, wherein the pump comprises a pump input and a pump output; the pump input is in fluidic communication with the combining output and is configured to receive the combined stream; and the pump output is in fluidic communication with the reverse osmosis input and is configured to output the combined stream to the reverse osmosis input.

24. The flow-through system of any one of claims 21 to 23, wherein the source stream comprises a first concentration of the radionuclides, and further wherein the reverse osmosis unit comprises a membrane configured to separate the combined stream into the concentrate stream that comprises a second concentration of the radionuclides that is greater than the first concentration of the radionuclides, and the filtrate stream that comprises a third concentration of the radionuclides that is less than the second concentration of the radionuclides.

25. The flow-through system of any one of claims 21 to 24, further comprising a first sensor module positioned to measure the concentrate stream, and the first sensor module operable to measure a first salinity and a first flow of the concentrate stream.

26. The flow-through system of claim 25, wherein the first sensor module is positioned downstream from the detection portion output.

27. The flow-through system of claim 25 or claim 26, further comprising a second sensor module positioned to measure the source stream, and the second sensor module operable to measure a second salinity and a second flow of the source stream.

28. The flow-through system of claim 27, further comprising a third sensor module positioned to be in contact with the filtrate stream, and the third sensor module operable to measure a third salinity and a third flow of the filtrate water stream.

29. The flow-through system of any one of claims 25 to 28, further comprising a controller that is in data communication with at least the first sensor module and the spectrometer, and is configured to transmit radioactivity data associated with the concentrate stream.

30. The flow-through system of claim 29, wherein the controller comprises memory that stores executable instructions, the executable instructions comprising:
obtain the measurement of radionuclides in the amount of the concentrate stream within the detection portion, and
compute a radioactivity value corresponding to the source stream by at least applying a scaling factor to the measurement of radionuclides corresponding to the concentrate stream.

31. The flow-through system of claim 30, wherein the scaling factor comprises a salinity ratio between the concentrate stream and the source stream.

32. The flow-through system of claim 30, wherein the executable instructions further comprise: detecting a steady state associated with at least total dissolved solids in the concentrate stream.

33. The flow-through system of any one of claims 21 to 32, wherein the spectrometer is a gamma spectrometer.

34. The flow-through system of claim 33, wherein the gamma spectrometer is a cadmium zinc telluride (CZT) semiconductor-based device.

35. The flow-through system of any one of claims 21 to 34, wherein the detection portion comprises a vessel defining therein a vessel interior through which the amount of the concentrate stream in the detection portion flows through, and the spectrometer is at least partially positioned within the vessel interior.

36. The flow-through system of any one of claims 21 to 35, further comprising a pressure valve that is operable to interact with the concentrate stream, including controlling a volume of the concentrate stream that is recirculated back into the combining portion.

37. The flow-through system of claim 36, wherein the pressure valve is positioned downstream from the detection output and upstream from the combining input.

38. The flow-through system of claim 37, further comprising an overflow valve that is operable to interact with the concentrate stream, and an input of the overflow valve is positioned upstream from an input of the pressure valve.

39. The flow-through system of any one of claims 21 to 38, further comprising a body, and at least the reverse osmosis unit, the detection portion, the spectrometer, and the combining portion are supported by the body.

40. The flow-through system of claim 39, wherein the body is portable and one or more wheels are mounted to the body.
